(19) United States
(12) Patent Application Publication
(10) Pub. No.: US 2025/0066409 A1
(43) Pub. Date: Feb. 27, 2025

(54) DERIVATIVE OF PENTACYCLIC TRITERPENOIDAL SAPONIN AND ITS PREPARATION METHOD AND APPLICATION

(71) Applicant: SOOCHOW UNIVERSITY, Suzhou (CN)

(72) Inventors: Yanli LIU, Suzhou (CN); Qiongming Xu, Suzhou (CN)

(21) Appl. No.: 18/942,353

(22) Filed: Nov. 8, 2024

Related U.S. Application Data
(63) Continuation of application No. PCT/CN2022/126911, filed on Oct. 24, 2022.

(30) Foreign Application Priority Data
Sep. 30, 2022 (CN) 202211211016.8

(51) Int. Cl. C07H 15/256; A61K 31/704

(52) U.S. Cl. CPC C07H 15/256; A61K 31/704

(57) ABSTRACT

Provided are a derivative of pentacyclic triterpenoidal saponin, a preparation method therefor, and a use thereof. A compound A3 is used as a raw material, a derivative is prepared by targeting a carboxyl group of C-28 and using an esterification reaction or amidation reaction, and said derivative is used as an active ingredient to prepare drugs having anti-inflammatory, antioxidant and anti-apoptotic effects. The derivative of pentacyclic triterpenoidal saponin does not show obvious cytotoxicity to macrophages, has better anti-inflammatory activity, and can alleviate the symptoms of colitis in mice. In addition, said derivative antagonizes the cytotoxicity of cisplatin and can significantly inhibit the release of reactive oxygen species (ROS) in renal cells caused by cisplatin.

This application is a Continuation of PCT/CN2022/126911, filed on Oct. 24, 2022, which claims priority to Chinese Patent Application No. 202211211016.8, filed on Sep. 30, 2022, which is incorporated by reference for all purposes as if fully set forth herein.

### TECHNICAL FIELD

The invention belongs to the technical field of chemical drug synthesis, and relates to a derivative of pentacyclic triterpenoidal saponin and its preparation method, and the application of the derivative in preparing anti-inflammatory (including inflammatory bowel disease) drugs.

### BACKGROUND OF THE INVENTION

Humans have been made a research on inflammation for a long time, and inflammation is a widespread and frequent disease. The normal inflammatory response is a defense process of the body and a biological response of the immune system to harmful stimuli (such as viruses, bacterial infections, toxins, toxic compounds, tissue damage). Inflammatory reactions arc mostly related to immune mechanisms and the immune cells involved include macrophages, B cells, T cells, NK cells, etc. Among them, macrophages are the key cells to initiate inflammation, and they participate in inflammatory reactions by activating the body's immune system. At present, the clinical treatment for inflammation is mainly the drug therapy. The common anti-inflammatory drugs are mainly divided into steroids (SAIDs) and non-steroids (NSAIDs), while the non-steroidal anti-inflammatory drugs are the most widely used anti-inflammatory drugs in the world. Although they have powerful anti-inflammatory, analgesic and antipyretic functions, they also have strong toxic side effects.

Inflammatory bowel disease (IBD) is a chronic inflammatory disease of the intestine, which clinically includes Crohn's disease (CD) and Ulcerative Colitis (UC). The main clinical manifestations are abdominal pain, watery diarrhea, blood in the stool and weight loss, as well as the influx of neutrophils and macrophages produces a large number of cytokines, proteolytic enzymes and free radicals, which leads to inflammation and ulcers. Studies have shown that the pathogenesis of IBD is related to genetic susceptibility, gut microbiota, living environment and immune abnormalities. At present, the clinical treatment drugs for IBD include: aminosalicylates, corticosteroids, immunomodulators, monoclonal antibodies, etc. However, these drugs have poor efficacy in some patients and often cause serious side effects. In summary, it is urgent to develop new drugs with high anti-inflammatory activity and low toxic side effects.

### TECHNICAL PROBLEMS

The existing anti-inflammatory drugs have obvious toxic side effects, and some drugs have the defect of short half-life. In order to solve this problem, the invention discloses a derivative of pentacyclic triterpenoidal saponin and its preparation method, and the application of the derivative in the preparation of anti-inflammatory (including inflammatory bowel disease) drugs.

### TECHNICAL SOLUTIONS

The present invention adopts the following technical solution: a derivative of pentacyclic triterpenoidal saponin has the following chemical structure formula:

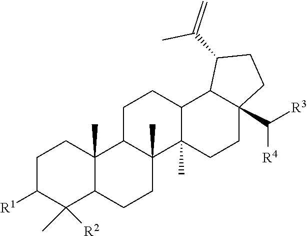

R¹ is hydrogen, hydroxy, halogen, C₁₋₈ alkoxy or —O-T, wherein T is a C₄₋₇ monosaccharide, including but not limited to glucose, arabinose, rhamnose, galactose or xylose or acetylated forms; Or T is a disaccharide including but not limited to, α-L-rhamnopyranosyl-(1→2)-α-L-arabinopyranosyl or acetylated forms; R² is alkyl, haloalkyl, oxygen-substituted alkyl; R³ is oxygen, hydroxy or hydrogen; R4 is amino, —NHR^{a}, —N(R^{a})₂, —OR^{b}, wherein R^{a} and R^{b} are respectively alkyl, aryl-C₁₋₆ alkyl, hydroxy-C₁₋₆ alkyl or amino-C₁₋₆ alkyl, cycloalkyl, aromatic heterocycle, amino acid or amino acid ester; Wherein R^{a} and R^{b} may also be linked with the following substituents by nucleophilic substitution reaction, amidation or esterification reaction, including alkoxy group, acyloxy group such as pyruvate acyloxy group and the like, alkylamino group, amide group such as oxaloacetate amido group and the like; Wherein the amino acids include glycine, aminobutyric acid, aminocaproic acid, phenylalanine, alanine, cysteine, leucine or serine; Amino acid esters include ethyl glycine, ethyl aminobutyrate, methyl aminocaproate, ethyl phenylalanine, ethyl alanine, ethyl cysteine, ethyl leucine, or ethyl serine.

The invention discloses the application of the derivative of pentacyclic triterpenoidal saponin in the preparation of anti-inflammatory drugs, antioxidant drugs and anti-apoptosis drugs.

The invention discloses the application of the derivative of pentacyclic triterpenoidal saponin in the preparation of a medicament for treating inflammatory bowel disease.

The present invention discloses a pharmaceutical composition comprising the derivative of pentacyclic triterpenoidal saponin as an active ingredient, and further comprising a pharmaceutically acceptable carrier. The active ingredient and pharmaceutical composition are used for the preparation of the medicament for the treatment of inflammatory diseases.

In the present invention, a pharmaceutically acceptable carrier refers to one or more compatible solid or liquid fillers or gel substances, which can be used for medicine with sufficient purity and low toxicity, and the components of the pharmaceutical composition intermingle with each other and with the active ingredient of the present invention without reducing the efficacy of the active ingredient. Some examples of pharmaceutically acceptable carriers are cellulose and its derivatives (e.g. sodium carboxymethyl cellulose, sodium ethyl cellulose, cellulose acetate, etc.), gelatin, talc, solid lubricants (e.g. stearic acid, magnesium stearate), calcium sulfate, vegetable oils (e.g. soybean oil, sesame oil, peanut oil, olive oil, etc.), polyols (e.g. propylene glycol, glycerol, mannitol, sorbitol, etc.), cyclodextrins (e.g. hydroxypropyl beta-cyclodextrins), emulsifiers (e.g. Tween), wetting agents (e.g. sodium lauryl sulfate, etc.), colorants, seasonings, stabilizers, antioxidants, preservatives, pyrogen-free water, etc.

The anemoside A3 derivative disclosed for the first time in the present invention shows no obvious cytotoxicity to THP-1 macrophages, can increase the level of IκB protein in NF-κB signal, and compared with the positive drug, the level of IκB protein is significantly increased (p<0.05), and the release of IL-6 and TNF-α is significantly reduced. These results suggest that the derivative of the present invention has better anti-inflammatory activity; The therapeutic effect on colitis mice shows that the derivatives of the present invention can alleviate the symptoms of colitis in mice. Furthermore, the main characteristic of nephrotoxicity of cisplatin is that it causes renal cell damage and apoptosis, and the derivative of the present invention can antagonize the cytotoxicity of cisplatin, and can obviously inhibit the release of reactive oxygen species (ROS) in renal cells caused by cisplatin.

### BRIEF DESCRIPTION OF THE DRAWINGS

FIG. 1 is a schematic diagram of the preparation of a derivative of pentacyclic triterpenoidal saponin of the present invention.

FIG. 2 is a schematic diagram of the preparation of a derivative of pentacyclic triterpenoidal saponin of the present invention.

FIG. 3 is a schematic diagram of the preparation of derivative of pentacyclic triterpenoidal saponins of the present invention.

FIG. 4 is a schematic diagram of the preparation of derivative of pentacyclic triterpenoidal saponins of the present invention.

FIG. 5 is a specific structure of a derivative of pentacyclic triterpenoidal saponin of the present invention.

FIG. 6 is a specific structure of a derivative of pentacyclic triterpenoidal saponin of the present invention.

FIG. 7 is a specific structure of a derivative of pentacyclic triterpenoidal saponin of the present invention.

FIG. 8 shows the effect of compound A3 and its derivatives on THP-1 cytotoxicity.

FIG. 9 is a Western blotting strip.

FIG. 10 shows Western blotting for detection of IκB protein expression.

FIG. 11 shows the release of inflammatory factors of compound A3-6.

FIG. 12 shows the effect of A3-6 on the DAI score of DSS-induced colitis mice.

FIG. 13 shows the effect of A3-6 on colon length in DSS-induced colitis mice.

FIG. 14 shows the in vitro anti-apoptotic effect of A3-6.

FIG. 15 shows the in vitro antioxidant effect of A3-6.

### EMBODIMENT OF THE INVENTION

The invention discloses the application of a derivative of pentacyclic triterpenoidal saponin in the preparation of an anti-inflammatory drug. The derivatives of the present invention may be administered alone or in combination with other therapeutic drugs. The mode of administration of the active ingredient or the pharmaceutical composition of the present invention is not particularly limited, and representative modes of administration include external, oral, rectal, parenteral (such as intravenous, intramuscular, or subcutaneous), and the like. Solid dosage forms for oral administration include capsules, tablets, pills, powders and granules; Liquid dosage forms for oral administration include pharmaceutically acceptable emulsions, solutions, suspensions, syrups or tinctures. In addition to the active ingredient, the liquid dosage form may contain inert diluents conventionally used in the art, such as water or other solvents, solubilizers and emulsifiers, such as ethanol, isopropanol, ethyl carbonate, ethyl acetate, propylene glycol, 1,3-butanediol, dimethylformamide and oils, in particular cottonseed oil, peanut oil, corn germ oil, olive oil, castor oil, sesame oil or mixtures of these substances and the like. In addition to these diluents, the compositions may also contain auxiliaries such as wetting agents, emulsifying and suspending agents, sweetening agents, corrigents and flavoring agents. In addition to that active ingredient, the suspension may contain suspending agent, such as cthoxylated isoctadecanol, polyoxyethylene sorbitan, dehydrated sorbitan esters, microcrystalline cellulose, aluminum methoxide and agar, or mixtures of these. Compositions for parenteral injection may comprise physiologically acceptable sterile aqueous or anhydrous solutions, dispersions, suspensions or emulsions, and sterile powders for redissolving into sterile injectable solutions or dispersions. Suitable aqueous and non-aqueous carriers, diluents, solvents or excipients include water, ethanol, polyols, and suitable mixtures thereof.

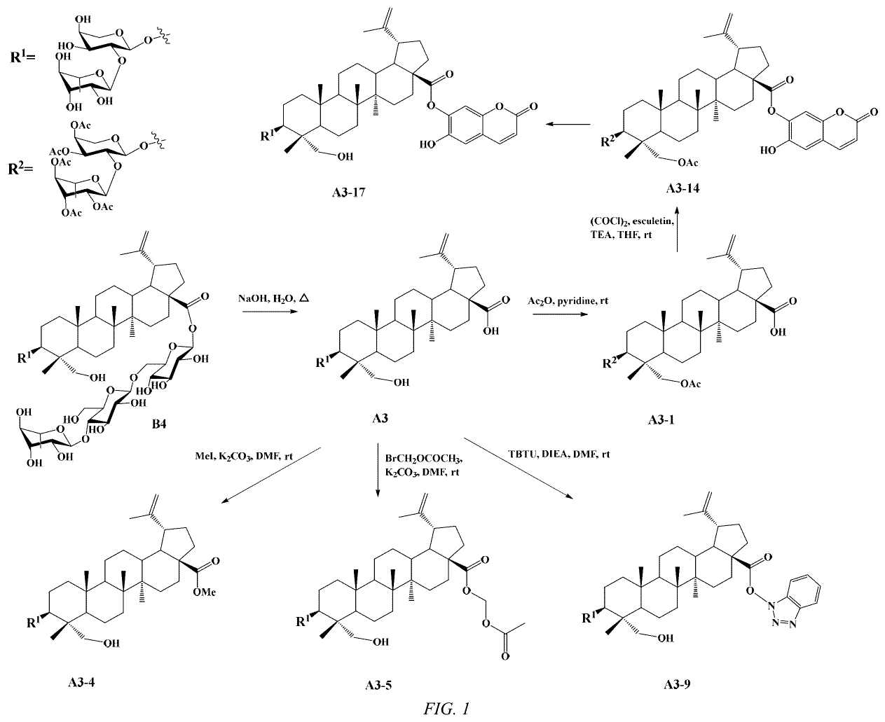

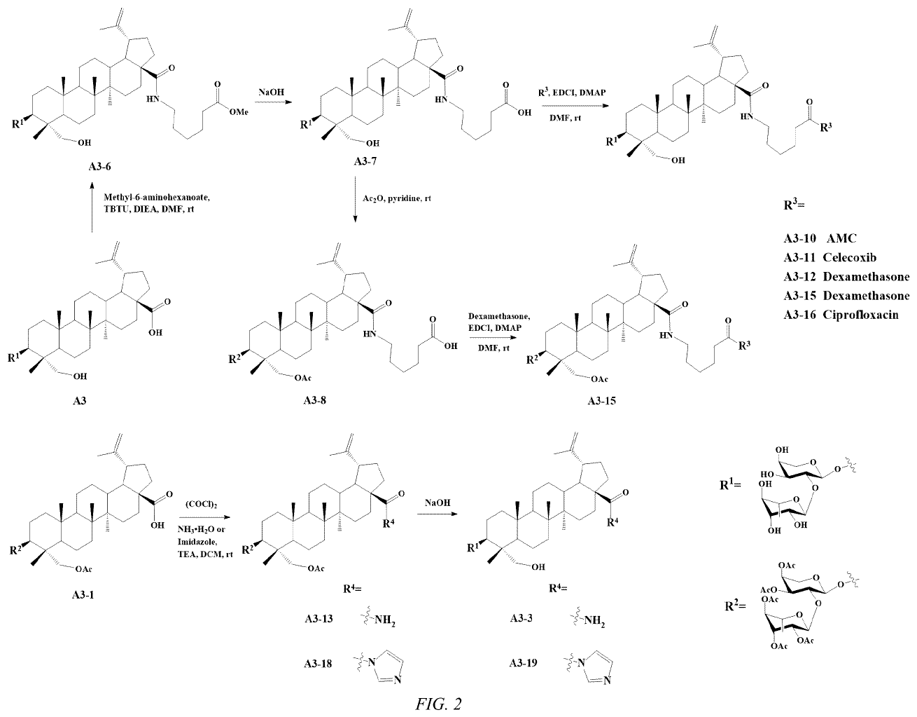

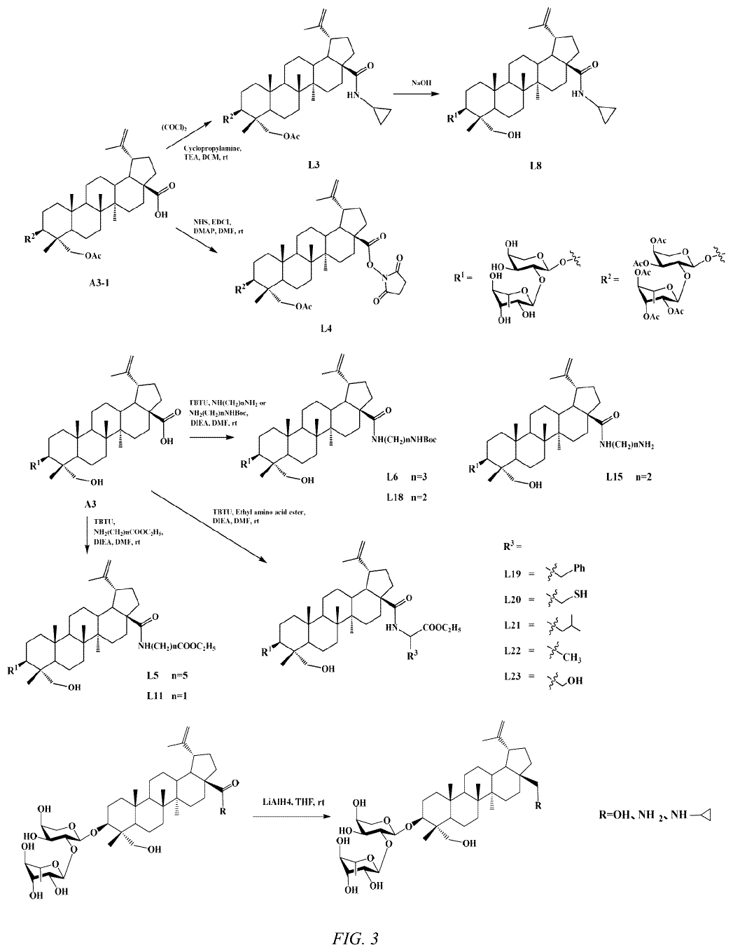

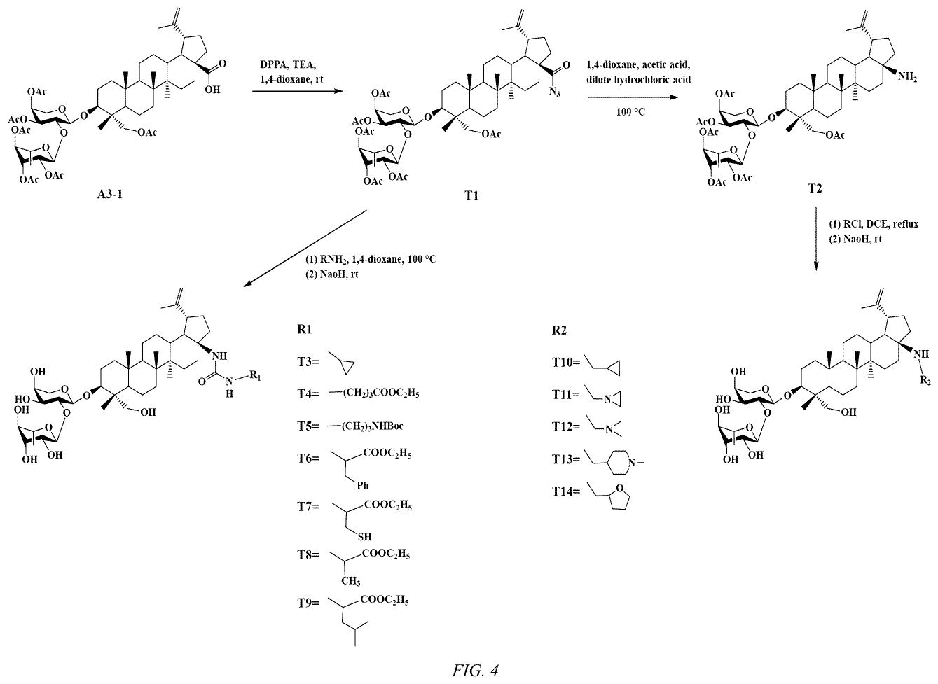

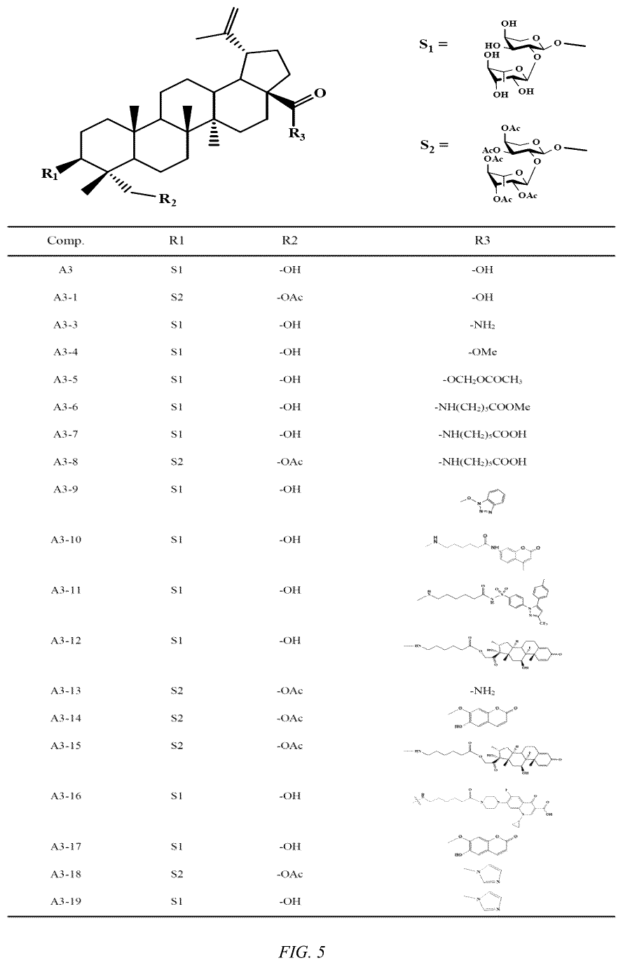

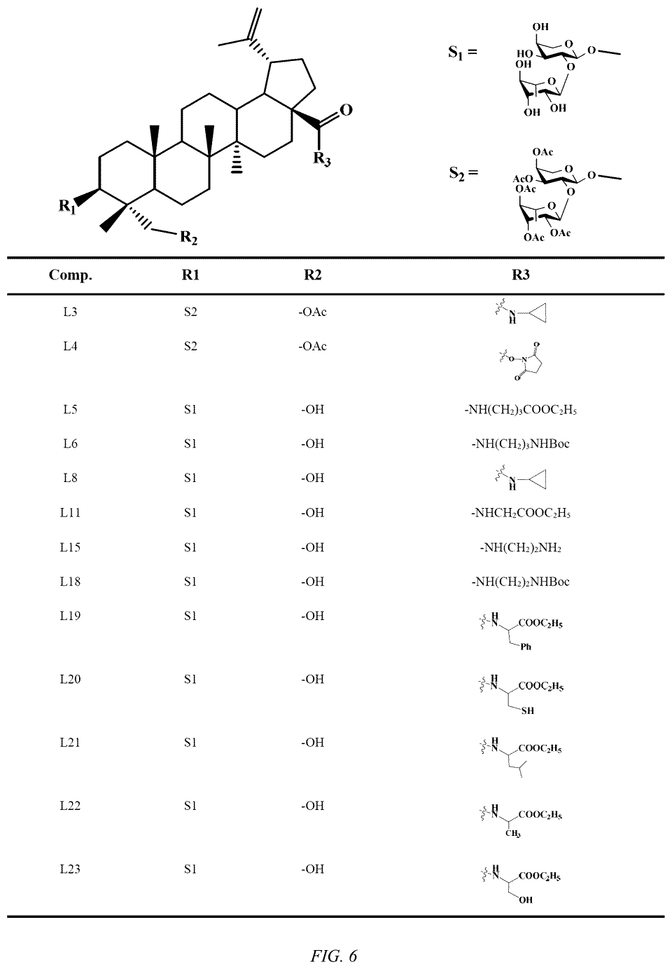

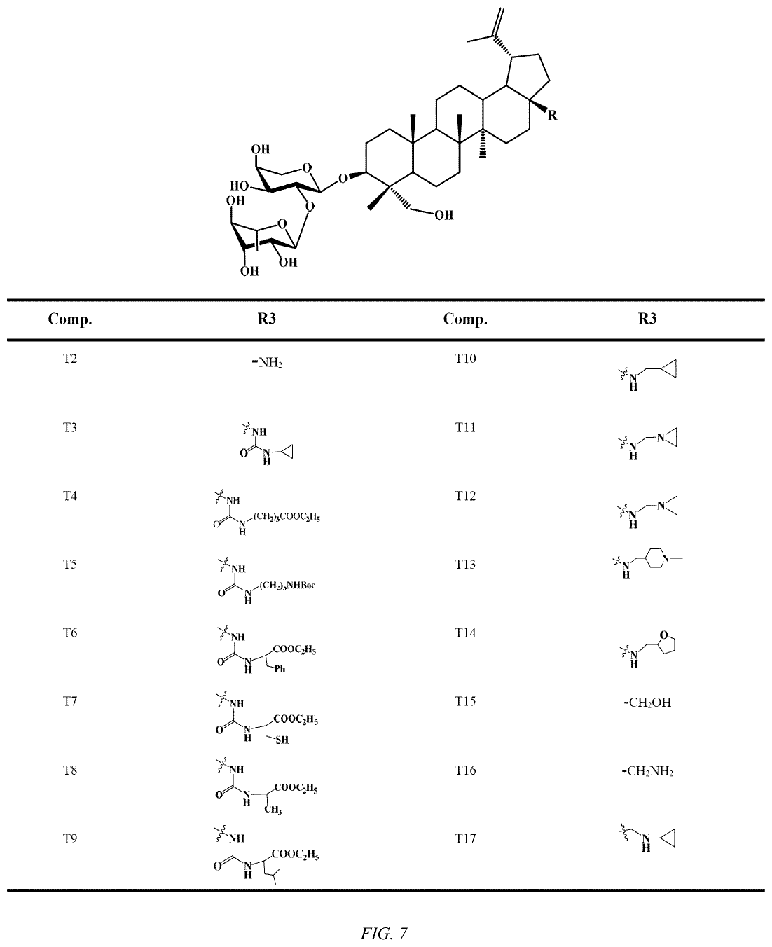

The prior art discloses that anemoside B4 (B4) has anti-inflammatory application and is considered to be the substance with the best anti-inflammatory effect among Pulsatilla saponins, but anemoside B4 is a pentasaccharide saponin with a molecular weight as high as 1220, extremely strong water solubility, short half-life and low oral availability, thus limiting its clinical application; Anemoside A3 is a triterpenoid saponin, a disaccharide saponin with small molecular weight and increased fat solubility, but anemoside A3 has no significant anti-inflammatory activity and has certain toxicity, such as hemolysis. The present invention carries out structural modification to obtain compounds with better anti-inflammatory activity and very low toxicity, especially with unexpectedly better activity than B4. The synthetic route of the derivative of pentacyclic triterpenoidal saponin of the present invention is shown in FIGS. 1 to 4, and the structure of the specific derivative is shown in FIGS. 5 to 7. The present invention will be further described below with reference to specific examples. It is to be understood that these examples are merely used to illustrate the invention and are not intended to limit the scope of the invention. Unless otherwise specified, percentages and parts are percentages by weight and parts by weight. Unless otherwise defined, all technical and scientific terms used herein have the same meanings as those familiar to those skilled in the art. Furthermore, any methods and materials similar or equivalent to those described can be used in the methods of the present invention. In the following preparation examples, the agents were mainly provided by Shanghai Chemical Reagent Company; TLC thin layer chromatography silica gel plate was produced by Shandong Yantai Jiangyou Silica Gel Development Company, model HSGF 254, and the normal phase column chromatography silica gel used for compound purification was produced by Beijing InnoChem Science & Technology Co., Ltd., with 200-300 mesh. The NMR was recorded with a Varian Mercury 400M nuclear magnetic resonance instrument, and the chemical shifts were expressed in δ (ppm).

The abbreviations of the present invention correspond to the following Chinese words: DMF: N,N-dimethylformamide; DCM: dichloromethane; THF: tetrahydrofuran; TBTU: O-benzotriazole-N,N,N′,N′-tetramethylurca tetrafluoroborate; EDCI: 1-(3-dimethylamino-propyl)-3-ethylcarbodiimide hydrochloride; DMAP: 4-dimethylaminopyridine; DIPEA (DIEA): N, N-diisopropylethylamine; TEA: triethylamine; Mel: methyl iodide; AMC: 7-amino-4-methylcoumarin.

### Example 1

The raw material anemoside B4 (25 g, 20.5 mmol) was dissolved in 130 mL of an aqueous solution of sodium hydroxide (1.9 g, 47.17 mmol), heated at 105° C. and refluxed for 10 h, during which the aqueous solution of sodium hydroxide was supplemented to adjust the pH=11˜12. After the reaction was completed, the reaction solution was centrifuged at 5000 rpm for 5 min, the precipitate was washed with water twice, filtered by suction, and dried to obtain 14.2 g of a light yellow solid, which was identified to be anemoside A3, and was then used as raw material for the preparation of the following derivatives. Purity 94%, a yield: 92.2%. ¹H-NMR (400 MHZ, MeOD): δ 5.18 (1H, brs, H-1 of rha), 4.59 (1H, d, J=5.0 Hz, H-1of ara), 4.72, 4.57 (each 1H, brs, H2-29), 3.93 (1H, m, H-3), 1.71 (3H, s, H-30), 1.27 (3H, d, J=6.2 Hz, H-6 of rha), 1.04 (3H, s, H-26), 1.03 (3H, s, H-24), 0.92 (3H, s, H-27), 0.70 (3H, s, H-25). ¹³C-NMR (125 MHz, MeOD): δ 184.09, 153.04, 109.18, 104.04, 101.64, 82.17, 76.40, 73.73, 73.41, 71.92, 71.83, 69.96, 68.88, 64.46, 64.39, 58.65, 51.95, 50.73, 43.85, 43.51, 41.74, 39.77, 39.18, 38.95, 37.63, 34.93, 34.47, 32.02, 30.98, 26.95, 26.52, 22.04, 19.53, 18.63, 17.75, 17.06, 16.79, 14.92, 13.31.

### Example 2

Anemoside A3 (2 g, 2.67 mmol) and acetic anhydride (3.27 g, 32 mmol) were dissolved in 17 mL of pyridine, the reaction was stirred at room temperature for 12 hours. After the reaction was completed, 40 mL of ethyl acetate was added, pH was adjusted to 4 with 10% dilute hydrochloric acid, the organic layer was washed three times with 50 mL of saturated salt water, dried with anhydrous sodium sulfate, filtered, concentrated under reduced pressure to remove the solvent. Then it was subjected to silica gel column chromatography (petroleum ether: ethyl acetate =3:1) to obtain 2.07 g of a white solid, a yield of 77%, namely A3-1. ESI-MS (m/z): 1002.4 [M−H]⁻, ¹H-NMR (300 MHz, DMSO-d₆): δ 12.08 (1H, s, COOH), 5.10 (1H, brs, H-1 of rha), 4.69, 4.56 (each 1H, brs, H2-29), 4.49 (1H, d, J=6.6 Hz, H-1of ara), 4.06 (1H, m, H-3), 2.09, 2.06, 2.06, 2.01, 1.94, 1.93 (each 3H, s, 6×CH3CO), 1.09 (3H, d, J=5.6 Hz, H-6 of rha), 1.65, 0.92, 0.86, 0.81, 0.72 (each 3H, s, 5×CH3).

### Example 3

Derivative A3-1 (250 mg, 0.25 mmol) was dissolved in 5 mL of anhydrous dichloromethane, and oxalyl chloride (0.15 mL, 1.25 mmol) was added and stirred at room temperature for 4 h. After the reaction was completed, the solvent was removed under reduced pressure to obtain a dry white solid, which was dissolved in 5 mL of anhydrous tetrahydrofuran, added dropwise into 10 mL of concentrated ammonia under ice bath cooling, stirred at room temperature for 2 h. After completion of the reaction, 50 mL of ethyl acetate was adjusted to pH=4 with 10% dilute hydrochloric acid, and the organic layer was washed three times with 50 mL of saturated salt water, dried with anhydrous sodium sulfate, filtered, and concentrated under reduced pressure to obtain 316 mg of white solid, namely A3-13. ¹H-NMR (400 MHZ, DMSO-d₆): δ 7.07, 6.62 (each 1H, s, CONH2), 5.10 (1H, brs, H-1 of rha), 4.65, 4.53 (each 1H, brs, H2-29), 4.49 (1H, d, J=6.9 Hz, H-1of ara), 4.09 (1H, m, H-3), 3.80, 3.77 (each 1H, d, J=12.1 Hz, H2-23) 2.09, 2.06, 2.05, 2.01, 1.94, 1.93 (each 3H, s, 6×CH3CO), 1.10 (3H, d, J=6.2 Hz, H-6 of rha), 0.90, 0.86, 0.82, 0.72 (each 3H, s, 5×CH3).

### Example 4

A white solid A3-13 (300 mg, 0.3 mmol) was dissolved in 10 mL of a mixed solution of methanol/tetrahydrofuran/water (2:1:1), and sodium hydroxide (108 mg, 2.7 mmol) was added and stirred at room temperature for 12 h. After the reaction was completed, the solvent was removed under reduced pressure, the salt was washed with 50 mL of water, and dried to give 170 mg of white solid with a yield of 75.5%, namely A3-3. ¹H-NMR (300 MHZ, DMSO-d₆): δ 7.07, 6.63 (each 1H, s, CONH2), 5.06 (1H, brs, H-1 of rha), 4.63, 4.59 (each 1H, brs, H2-29), 4.43 (1H, d, J=5.9 Hz, H-1of ara), 4.37 (1H, m, H-3), 1.07 (3H, d, J=6.2 Hz,H-6 of rha), 1.63, 0.91, 0.85, 0.78, 0.54 (each 3H, s, 5×CH3). ¹³C-NMR (125 MHz, DMSO): δ 178.25, 151.12, 109.42, 103.09, 100.04, 79.54, 74.31, 73.02, 72.19, 70.59, 70.53, 68.28, 67.97, 64.51, 62.60, 55.02, 50.26, 49.60, 46.63, 46.27, 42.50, 42.15, 40.40, 38.56, 37.85, 36.76, 36.32, 33.71, 32.73, 30.48, 29.11, 25.65, 25.45, 20.73, 19.20, 17.95, 17.24, 16.59, 16.07, 14.41, 12.97.

### Example 5

Anemoside A3 (150 mg, 0.2 mmol) was dissolved in 5 mL of DMF, potassium carbonate (83 mg, 0.6 mmol) was added, then Mel (28 mg, 0.2 mmol) was added at 0° C., and the reaction was carried out at room temperature for 24 h. After the reaction was completed, extracted with n-butanol, the organic layer was washed three times with 50 mL of saturated salt water, dried over anhydrous sodium sulfate, filtered, concentrated under reduced pressure to remove the solvent, and it was subjected to silica gel column chromatography (dichloromethane: Methanol=8:1) to obtain 88 mg of off-white solid with a yield of 58.6%, namely A3-4. ¹H-NMR (400 MHZ, pyridine-d₅): δ 6.02(1H, brs, H-1 of rha), 6.73, 6.59 (each 1H, brs, H2-29), 5.12 (1H, d, J=6.2 Hz, H-1of ara), 4.58 (1H, m, H-3), 3.72 (3H, COOCH₃), 1.71, 1.65, 1.07, 1.00, 0.95, 0.90 (each 3H, s, 6×CH3). ¹³C-NMR (125 MHz, pyridine-d₅): δ 176.25, 150.57, 109.90, 104.13, 101.48, 80.90, 75.66, 74.47, 73.91, 72.33, 72.14, 69.49, 69.09, 65.40, 63.71, 56.54, 51.10, 50.63, 49.53, 47.65, 47.32, 43.39, 42.44, 40.76, 39.01, 38.29, 36.85, 36.76, 34.10, 32.08, 30.69, 29.85, 26.17, 25.70, 20.90, 19.12, 18.32, 17.91, 16.68, 15.98, 14.60, 13.55.

### Example 6

Anemoside A3 (500 mg, 0.67 mmol) was dissolved in 8 mL of DMF, potassium carbonate (278 mg, 2.01 mmol) was added, stirred at room temperature for 30 min, bromomethyl acetate (205 mg, 1.34 mmol) was added, and reacted at room temperature for 24 h. After the reaction was completed, 20 mL of water was added, extracted with 30 mL of ethyl acetate three times, the organic layer was washed twice with 30 mL of saturated salt water, dried with anhydrous sodium sulfate, filtered, and concentrated under reduced pressure to remove the solvent, and it was subjected to silica gel column chromatography (dichloromethane: methanol=8:1) obtain 55 mg of off-white solid with a yield of 11%, namely A3-5. ¹H-NMR (400 MHZ, MeOD): δ 5.81, 5.73 (each 1H, d, J=5.7 Hz, OCH2O), 5.17 (1H, brs, H-1 of rha), 4.75, 4.63 (each 1H, brs, H2-29), 4.58 (1H, d, J=5.0 Hz, H-1of ara), 3.92 (1H, m, H-3), 2.10 (3H, s, COCH3), 1.72 (3H, s, H-30), 1.26 (3H, d, J=6.2 Hz, H-6 of rha), 1.04 (3H, s, H-26), 0.97 (3H, s, H-24), 0.92 (3H, s, H-27), 0.70 (3H, s, H-25). ¹³C-NMR (125 MHZ, pyridine-d₅): δ 175.76, 170.92, 151.40, 110.21, 104.07, 101.69, 82.06, 80.04, 76.48, 73.74, 73.43, 71.96, 71.83, 69.97, 68.88, 64.50, 64.38, 57.69, 51.70, 50.35, 43.84, 43.43, 41.71, 39.71, 39.48, 37.61, 37.36, 34.78, 32.58, 31.62, 31.29, 30.53, 30.46, 26.63, 26.48, 21.89, 20.40, 19.34, 18.57, 17.76, 16.99, 16.50, 14.93, 13.30.

### Example 7

Anemoside A3 (5.0 g, 6.67 mmol), TBTU (3.2 g, 10 mmol) and DIPEA (2.6 g, 20 mmol) were dissolved in 50 mL of DMF, the reaction was stirred at room temperature for 4 hours, the reaction was monitored by TLC, methyl 6-aminocaproate hydrochloride (1.8 g, 10 mmol) was added, and the reaction was carried out for 12 hours. After the reaction was completed, the solvent was removed by concentration under reduced pressure, and the mixture was prepared by medium pressure (methanol: water=75:25) to obtain 5.2 g of an off-white solid with a yield of 89.6%, namely A3-6. ¹H-NMR (400 MHz, CD₃OD): δ 7.61(1H, t, NH), 5.17 (1H, brs, H-1 of rha), 4.57 (1H, d, J=4.8 Hz, H-1of ara), 4.72, 4.60 (each 1H, brs, H2-29), 3.86 (1H, m, H-3), 3.11 (1H, m, H-31), 2.59 (1H, m, H-35), 3.67 (3H, s, COOCH3), 1.71 (3H, s, H-30), 1.03(3H, s, H-26), 0.98 (3H, s, H-24), 0.91 (3H, s, H-27), 0.69 (3H, s, H-25), 1.26 (3H, d, J=6.2 Hz, H-6 of rha). ¹³C-NMR (125 MHz, CD3OD): δ 179.14, 175.87, 152.44, 109.95, 104.35, 101.93, 82.31, 76.69, 73.97, 73.71, 72.17, 72.06, 70.20, 69.17, 64.80, 64.60, 57.00, 52.09, 52.03, 51.48, 48.16, 44.09, 43.63, 42.01, 40.00, 39.48, 38.98, 37.86, 35.08, 34.77, 34.24, 32.00, 30.63, 30.44, 27.61, 27.05, 26.74, 25.77, 22.24, 19.69, 18.83, 18.00, 17.26, 16.90, 15.11, 13.55.

### Example 8

A3-6 (5.0 g, 5.7 mmol) was dissolved in 24 mL of a mixed solution of methanol/tetrahydrofuran/water (2:1:1), sodium hydroxide (683 mg, 17.1 mmol) was added, and stirred at room temperature for 12 h. After the reaction was completed, the pH was adjusted to 4 with 10% dilute hydrochloric acid, the solvent was concentrated under reduced pressure to remove the solvent, the salt was washed off with water, and dried to obtain 4.4 g of a white solid of a yield of 89%, namely A3-7. ESI-MS m/z: 864.4 [M] ¹H-NMR (400 MHz, CD₃OD): δ 7.57(1H, t, NH), 5.15 (1H, brs, H-1 of rha), 4.55 (1H, d, J=5.0 Hz, H-1of ara), 4.69, 4.57 (each 1H, brs, H2-29), 3.89 (1H, m, H-3), 3.25 (1H, m, H-31), 2.57 (1H, m, H-35), 1.68 (3H, s, H-30), 1.01 (3H, s, H-26), 0.96 (3H, s, H-24), 0.89 (3H, s, H-27), 0.67 (3H, s, H-25), 1.23 (3H, d, J=6.2 Hz, H-6 of rha). ¹³C-NMR (125 MHZ, CD₃OD): δ 179.13, 179.05, 178.23, 152.46, 109.92, 104.31, 101.92, 82.33, 76.70, 73.97, 73.68, 72.18, 72.06, 70.21, 69.14, 64.75, 64.62, 56.98, 52.10, 51.50, 48.15, 44.09, 43.63, 42.01, 40.06, 39.97, 39.49, 38.98, 37.86, 35.48, 35.08, 34.23, 32.01, 30.62, 30.47, 27.73, 27.06, 26.74, 26.04, 22.24, 19.70, 18.83, 17.99, 17.26, 16.89, 15.12, 13.55.

### Example 9

A3-7 (2.0 g, 2.3 mmol) and acetic anhydride (2.8 g, 27.8 mmol) were dissolved in 15 mL of pyridine, the reaction was stirred at room temperature for 12 hours. After the reaction was completed, 100 mL of ethyl acetate was added, the pH was adjusted to 4 with 10% dilute hydrochloric acid, the organic layer was washed three times with 50 mL of saturated salt water, dried over anhydrous sodium sulfate, filtered, concentrated under reduced pressure to remove the solvent, and it was subjected to silica gel column chromatography (petroleum ether: Ethyl acetate=8:1) to obtain 1.3 g of a white solid with a yield of 50.7%, namely A3-8. ¹H-NMR (400 MHZ, CDCl₃): δ 7.27(1H, t, NH), 5.30 (1H, brs, H-1 of rha), 4.73, 4.59 (each 1H, brs, H2-29), 4.42 (1H, d, J=4.6 Hz, H-1of ara), 2.14, 2.11, 2.10, 2.05, 2.03, 1.97 (each 3H, s, 6×CH₃CO), 1.21 (3H, d, J=6.2 Hz, H-6 of rha), 1.68, 0.94, 0.92, 0.85, 0.78(each 3H, s, 5×CH₃).

### Example 10

Anemoside A3 (200 mg, 0.27 mmol), TBTU (128 mg, 0.4 mmol) and DIPEA (52 mg, 0.4 mmol) were dissolved in 6 mL of DMF, and the reaction was stirred at room temperature for 3 h. After the reaction was completed, 20 mL of dichloromethane was added, the organic layer was washed three times with 50 mL of water and with 50 mL of saturated salt water once, dried over anhydrous sodium sulfate, filtered, concentrated under reduced pressure to remove the solvent, and it was subjected to silica gel column chromatography (dichloromethane: methanol=10:1) to obtain 148 mg of off-white solid with a yield of 63%, namely A3-9. ESI-MS m/z: 868.5. ¹H-NMR (300 MHz, DMSO-d₆): δ 8.17, 7.62 (each 1H, d, J=8.4 Hz, H2-Ar), 7.70, 7.54 (each 1H, t, J=7.6Hz, H2-Ar), 5.75 (1H, brs, H-1 of rha), 4.72, 4.40 (each 1H, brs, H2-29), 4.45, 4.33 (each 1H, d, J=10.8 Hz, H2-23), 4.40 (1H, m, H-3), 1.07 (3H, d, J=6.0 Hz,H-6 of rha), 1.69, 1.02, 0.88, 0.76, 0.55 (each 3H, s, 5×CH3). ¹³C-NMR (125 MHz, DMSO-d₆): δ 172.11, 149.24, 142.94, 129.62, 128.41, 125.44, 120.19, 110.51, 108.47, 103.02, 100.00, 79.41, 74.29, 72.92, 72.12, 70.53, 70.46, 68.23, 67.89, 64.42, 62.52, 56.66, 49.93, 49.19, 46.52, 46.51, 42.44, 42.21, 40.28, 38.43, 38.19, 36.21, 35.83, 33.45, 30.57, 29.88, 29.63, 25.55, 25.01, 20.43, 19.04, 17.88, 17.12, 16.44, 15.74, 14.50, 12.89.

### Example 11

A3-7 (500 mg, 0.58 mmol) and AMC (59.5 mg, 0.46 mmol) were dissolved in 10 mL of DMF, stirred at room temperature for 10 min, then EDCI (275 mg, 1.45 mmol) was added. After reacting at room temperature overnight, the reaction was completed, extracted with 50mL of dichloromethane three times, washed with 100 ml of water once, dried with anhydrous sodium sulfate, filtered, concentrated under reduced pressure to remove the solvent, and it was subjected to silica gel column chromatography (dichloromethane: methanol=8:1) to obtain 35 mg of white solid with a yield of 7%, namely A3-10. In the preparation schematic diagram of the attached drawing, for convenience of understanding, the corresponding substituent groups of raw materials and products are represented by R³, which does not affect the understanding of those skilled in the art. ESI-MS m/z: 1021.9 [M] ¹H-NMR (400 MHZ, CD₃OD): δ 7.57(1H, t, H28-NH), 7.81, 7.69 (each 1H, d, J=2.0 Hz, H8, H5-AMC), 7.48 (1H, dd, J=8.6, 2.0 Hz, H6-AMC), 6.23 (1H, d, J=1.2 Hz, H3-AMC), 5.15 (1H, brs, H-1 of rha), 4.67, 4.55 (each 1H, brs, H-29), 3.89 (1H, m, H-3), 2.45 (3H, d, J=1.2 Hz, H4-AMC), 1.24 (3H, d, J=6.2 Hz, H-6 of rha), 1.65, 0.95, 0.90, 0.82, 0.60 (each 3H, s, 5×CH3). 13C-NMR (125 MHZ, CD₃OD): δ179.17, 174.81, 163.37, 155.45, 155.35, 152.40, 143.90, 126.69, 117.01, 116.98, 113.48, 109.92, 107.80, 104.25, 101.87, 82.24, 76.61, 73.96, 73.66, 72.17, 72.05, 70.18, 69.11, 64.71, 64.58, 56.98, 52.06, 51.42, 49.30, 48.11, 44.01, 43.59, 41.96, 39.96, 39.48, 38.95, 37.88, 37.80, 35.05, 34.23, 31.97, 30.77, 30.57, 30.42, 27.60, 27.04, 26.68, 26.20, 22.20, 19.62, 18.75, 18.61, 18.01, 17.23, 16.84, 15.07, 13.52.

### Example 12

A3-7 (200 mg, 0.23 mmol) and celecoxib (73.5 mg, 0.19 mmol) were dissolved in 4 mL of DMF, stirred at room temperature for 10 min, then EDCI (110 mg, 0.58 mmol) was added. After reacting at room temperature overnight, the reaction was completed, and it was extracted with 50 mL of dichloromethane three times, washed with 100 mL of water once, dried with anhydrous sodium sulfate, filtered, concentrated under reduced pressure to remove the solvent, and it was subjected to silica gel column chromatography (petroleum ether: Ethyl acetate=8:1) to obtain 34 mg of a white solid with a yield of 14.3%, namely A3-11. ESI-MS m/z: 1227.9 [M+1]. ¹H-NMR (300 MHz, CD₃OD): δ 8.02 (2H, d, J=8.2 Hz, cele-H), 7.53 (2H, d, J=8.4 Hz, cele-H), 7.20-7.17 (4H, m, cele-H), 6.91 (1H, s, CH), 5.15 (1H, brs, H-1 of rha), 4.68, 4.56 (each 1H, brs, H2-29), 3.80 (1H, m, H-3), 2.36 (3H, s, cele-CH₃), 1.23 (3H, d, J=6.1 Hz, H-6 of rha), 1.67, 0.98, 0.91, 0.85, 0.66 (each 3H, s, 5×CH3). ¹³C-NMR (125 MHZ, CD₃OD): δ 179.12, 175.36, 152.49, 147.24, 144.42, 141.20, 139.34, 130.80, 130.30, 130.16, 127.19, 126.82, 125.59, 109.99, 107.21, 104.33, 101.98, 82.41, 76.78, 74.02, 73.68, 72.24, 72.11, 70.28, 69.15, 65.77, 64.68, 57.01, 52.14, 51.52, 48.50, 48.21, 44.14, 43.67, 42.06, 40.01, 39.86, 39.51, 39.04, 37.91, 37.60, 37.26, 35.59, 35.12, 34.26, 33.16, 32.06, 30.99, 30.84, 30.71, 30.56, 30.36, 29.78, 27.40, 27.11, 26.78, 25.45, 23.83, 22.29, 21.48, 19.75, 18.91, 18.07, 17.34, 16.95, 15.16, 14.54, 13.62.

### Example 13

The preparation method of A3-12 was similar to that of A3-11, except that the raw material to obtain the substituent R³ was replaced, and the yield was 19.5%. ¹H-NMR (400 MHz, CD₃OD): δ 7.57(1H, brs, NH), 7.42, 6.29 (each 1H, d, J=10.2 Hz, Dex-H1, H2), 6.08 (1H, s, Dex-H4), 5.14 (1H, brs, H-1 of rha), 4.95 (1H, s, Dex-H17), 4.70, 4.58 (each 1H, brs, H2-29), 4.60 (1H, s, H-1of ara), 4.26 (1H, m, Dex-H11), 3.80 (1H, m, H-3), 3.59 (1H, m, H-31), 2.57 (1H, m, H-35), 2.11 (1H, m, Dex-H16), 1.85 (2H, m, Dex-H6), 1.75 (1H, s, Dex-H8), 1.54-1.50 (4H, m, Dex-H12, H15), 1.68 (3H, s, H-30), 1.59 (3H, s, Dex-H19), 1.36-1.30 (3H, m, Dex-H7, H14), 1.23 (3H, d, J=6.0 Hz, H-6 of rha), 1.01 (3H, s, H-26), 0.97 (3H, s, H-24), 0.89 (3H, s, H-27), 0.86 (3H, d, J=6.0 Hz, Dex-H22), 0.67 (3H, s, H-25). ¹³C-NMR (125 MHZ, CD₃OD): δ 206.88, 189.09, 179.03, 174.99, 171.11, 156.05, 152.40, 129.78, 125.10, 109.91, 104.22, 102.94, 101.92, 101.78, 92.48, 82.33, 76.74, 73.91, 73.55, 73.08, 72.83, 72.13, 72.00, 70.19, 69.61, 69.03, 64.60, 56.94, 52.07, 51.46, 50.34, 50.19, 49.71, 48.42, 48.12, 45.08, 44.06, 43.60, 41.99, 39.92, 39.88, 39.46, 38.97, 37.83, 37.28, 37.17, 35.65, 35.52, 35.07, 34.63, 34.24, 33.39, 32.22, 31.99, 30.61, 30.32, 28.82, 27.42, 27.04, 26.69, 25.70, 23.70, 23.67, 22.22, 19.68, 18.84, 18.00, 17.26, 17.10, 16.90, 15.28, 15.10, 13.56.

### Example 14

Intermediate A3-1 (500 mg, 0.5 mmol) was dissolved in 5 mL of anhydrous dichloromethane, 0.5 mL of oxalyl chloride was added and stirred at room temperature for 4 h. After the reaction was completed, the solvent was removed under reduced pressure to obtain a dry white solid, which was dissolved in 3 mL of anhydrous tetrahydrofuran, dropwise added to a solution of aesculetin (71.2 mg, 0.4 mmol) in THF under an ice bath, and TEA (137 μL, 1 mmol) was added to react at room temperature for 36 h. After the reaction was completed, the solvent was removed under reduced pressure, and it was subjected to silica gel column chromatography (petroleum ether: ethyl acetate=2:1) to obtain 250 mg of a yellow solid with a yield of 43%, namely A3-14. ¹H NMR (400 MHZ, CDCl₃): δ 7.61, 6.29 (each 1H, d, J=9.5 Hz, H4, H3-Esculetin), 7.13, 6.99 (each 1H, s, H8, H5-Esculetin), 5.23 (1H, brs, H-1 of rha), 4.74, 4.63 (each 1H, brs, H2-29), 4.43 (1H, d, J=6.4 Hz, H-1 of ara), 3.02 (1H, m, H-3), 2.14, 2.11, 2.10, 2.06, 2.03, 1.97 (each 3H, s, 6×CH3CO), 1.21(3H, d, J=6.2 Hz, H-6 of rha), 1.71, 1.00, 0.97, 0.86, 0.78 (each 3H, s, 5×CH3).

### Example 15

The preparation method of A3-15 was similar to that of A3-11, except that the raw material to obtain the substituent R³ was replaced, and the yield was 34.8%. ¹H NMR (400 MHz, CDCl₃): δ 7.24, 6.35 (each 1H, d, J=10.2 Hz, Dex-H1, H2), 6.14 (1H, s, Dex-H4), 5.07 (1H, brs, H-1 of rha), 4.91 (1H, s, Dex-H17), 4.75, 4.61 (each 1H, brs, H2-29), 4.42 (1H, s, H-1 of ara), 4.14 (1H, m, Dex-H11), 3.89 (1H, m, H-3), 3.58 (1H, m, H-31), 2.63 (1H, m, H-35), 1.44-1.39 (4H, m, Dex-H12, H15), 1.57 (3H, s, Dex-H19), 2.16, 2.13, 2.12, 2.07, 2.05, 1.99 (each 3H, s, 6×CH3CO), 1.23(3H, d, J=6.2 Hz, H-6 of rha), 1.70, 1.08, 0.96, 0.87, 0.79 (each 3H, s, 5×CH3).

### Example 16

A3-7 (200 mg, 0.23 mmol) and Ciprofloxacin (83 mg, 0.25 mmol) were dissolved in 4 mL of DMF and EDCI (83.2 mg, 0.43 mmol) was added, and the reaction was carried out for 19 h. After the completion of the reaction, 50 mL of water was added and extracted with 50 mL of ethyl acetate three times to obtain 80 mg of crude precipitate, which was finally purified by semi-preparative HPLC (mobile phase, methanol: water=75:25, 100 mM ammonium formate) to obtain 10 mg of yellow solid with a yield of 5.6%, namely A3-16. ¹H NMR (300 MHZ, CD3OD): δ 8.83 (1H, s, Hb-alkene), 7.97 (2H, d, J=12.9 Hz, Hb-aryl), 7.61 (1H, s, NH), 5.15 (1H, brs, H-1 of rha), 4.70, 4.58 (each 1H, brs, H2-29), 4.12 (1H, m, HB-H1), 3.42 (1H, m, H-3), 1.24 (3H, d, J=6.1 Hz, H-6 of rha), 1.69, 1.01, 0.95, 0.84, 0.63 (each 3H, s, 5×CH3).

### Example 17

A3-14 (100 mg, 0.086 mmol) was dissolved in 3 mL of ammonia methanol solution and stirred at room temperature overnight. After completion of the reaction, the solvent was removed by concentration under reduced pressure, the salt was washed off with water, and dried to obtain 64 mg of yellow solid, namely A3-17. ¹H NMR (400 MHZ, CD3OD): δ7.85, 6.23 (each 1H, d, J=9.4 Hz, H4, H3-Esculetin), 7.22, 6.84 (each 1H, s, H8, H5-Esculetin), 5.08(1H, brs, H-1 of rha), 4.72, 4.61 (each 1H, brs, H2-29), 4.45 (1H, d, J=6.4 Hz, H-1 of ara), 4.09, 3.95 (each 1H, d, J=11.5 Hz, H2-23), 3.37 (1H, m, H-3), 1.72 (3H, s, H-30), 1.13 (3H, d, J=6.2 Hz, H-6 of rha), 1.05 (3H, s, H-26), 1.02 (3H, s, H-24), 0.81 (3H, s, H-27), 0.69 (3H, s, H-25). ¹³C-NMR (125 MHz, MeOD): δ 175.32, 172.31, 163.18, 155.00, 151.52, 145.37, 137.73, 122.50, 113.01, 112.50, 110.21, 104.38, 104.28, 101.84, 83.08, 76.74, 73.72, 72.83, 71.93, 71.83, 70.11, 68.44, 66.34, 64.03, 58.08, 52.00, 50.50, 49.30, 49.08, 43.44, 43.04, 41.88, 39.64, 39.40, 37.79, 37.79, 35.10, 32.91, 31.39, 30.65, 26.66, 26.40, 20.71, 19.34, 18.88, 17.86, 17.00, 16.59, 14.88, 13.02.

### Example 18

Intermediate A3-1 (500 mg, 0.5 mmol) was dissolved in 5 mL of anhydrous dichloromethane, 1 mL of oxalyl chloride, 2 drops of DMF were added and stirred at room temperature for 6 h. After the reaction was completed, the solvent was removed under reduced pressure to obtain a dry white solid, which was dissolved in 5 mL of anhydrous dichloromethane, imidazole (41 mg, 0.6 mmol) and triethylamine (61 mg, 0.6 mmol) were added, and reacted at room temperature overnight. After the reaction was completed, the solvent was removed by concentration under reduced pressure and it was subjected to silica gel column chromatography (petroleum ether: ethyl acetate=3:1) to obtain 350 mg of white solid, namely A3-18. ¹H-NMR (400 MHZ, CDCl₃): δ 7.71, 7.52 (each 1H, dd, J=5.7, 3.3 Hz, H-Imidazole), 5.24 (1H, brs, H-1 of rha), 4.73, 4.60 (each 1H, brs, H2-29), 4.42 (1H, d, J=6.4 Hz, H-1 of ara), 4.12 (1H, m, H-3), 2.13, 2.10, 2.09, 2.05, 2.03, 1.96 (each 3H, s, 6×CH3CO), 1.21 (3H, d, J=5.6 Hz, H-6 of rha), 1.68, 0.94, 0.90, 0.85, 0.77 (each 3H, s, 5×CH3). ¹³C-NMR (125 MHz, CDCl3): δ 176.72, 170.51, 170.45, 170.37, 170.23, 170.14, 169.71, 150.69, 131.01, 128.95, 109.69, 103.63, 98.24, 82.01, 77.44, 77.32, 77.12, 76.80, 74.39, 72.01, 71.16, 69.68, 68.71, 67.97, 67.21, 65.67, 65.24, 56.66, 51.38, 50.86, 49.58, 48.15, 47.06, 42.43, 42.08, 40.79, 38.72, 38.35, 37.05, 36.88, 34.14, 32.24, 30.69, 29.71, 25.83, 25.62, 21.13, 21.07, 21.03, 20.91, 20.87, 20.76, 19.50, 19.29, 18.07, 17.42, 16.71, 16.09, 14.64, 13.83, 12.61.

### Example 19

A white solid A3-18 (100 mg, 0.095 mmol) was dissolved in 4 mL of a mixed solution of methanol/tetrahydrofuran/water (2:1:1), sodium hydroxide (68.4 mg, 1.7 mmol) was added and stirred at room temperature overnight After the reaction was completed, the solvent was removed under reduced pressure, the salt was washed off with water, and dried to obtain 50 mg of white solid, namely A3-19. ¹H-NMR (400 MHZ, MeOD): δ 7.35, 7.12(each 1H, dd, J=8.6, 2.5 Hz, H-Imidazole), 5.05 (1H, brs, H-1 of rha), 4.62, 4.50 (each 1H, brs, H2-29), 4.45(1H, d, J=4.7 Hz, H-1 of ara), 3.80 (1H, m, H-3), 1.60 (3H, s, H-30), 1.13 (3H, d, J=4.9 Hz, H-6 of rha), 0.92 (3H, s, H-26), 0.84 (3H, s, H-24), 0.79 (3H, s, H-27), 0.58 (3H, s, H-25). ¹³C-NMR (125 MHz, MeOD): δ 177.99, 151.59, 110.10, 104.10, 101.69, 82.06, 76.47, 73.75, 73.46, 71.96, 71.84, 69.97, 68.92, 64.54, 64.38, 57.71, 51.71, 51.61, 50.46, 43.85, 43.41, 41.66, 39.72, 39.50, 37.69, 37.61, 34.77, 32.97, 31.45, 30.60, 26.65, 26.50, 21.89, 19.35, 18.58, 17.77, 16.99, 16.38, 14.96, 13.31.

### Example 20

The preparation method of L3 was similar to that of A3-6, except the methyl 6-aminohexanoate hydrochloride was replaced with the required raw material with a yield of 67.2%. 1H NMR (400 MHZ, CDC13) 8 8.02 (s, 1H), 5.75 (s, 1H), 5.25 (d, J=3.0 Hz, 1H), 4.97 (dd, J=8.8, 3.4 Hz, 1H), 4.73 (s, 1H), 4.59 (s, 1H), 4.42 (d, J=6.4 Hz, 1H), 3.95 (dd, J=12.9, 3.4 Hz, 1H), 2.64 (d, J=3.0 Hz, 1H), 2.14 (s, 4H), 2.10 (d, J=3.6 Hz, 6H), 2.04 (d, J=8.4 Hz, 6H), 1.97 (s, 3H), 1.68 (s, 4H), 1.21 (d, J=6.2 Hz, 3H), 1.11 (d, J=13.1 Hz, 2H), 0.93 (s, 6H), 0.86 (s, 3H), 0.78 (s, 3H), 0.43 (s, 2H).

### Example 21

THP-1 cells were seeded in a 96-well plate at 1×10⁴ cells/well, 100 μL/well, and cultured conventionally. After the cells reached 80%, they were induced with 100 ng/ml of phorbol ester (phorbol-12-Myristate-13-Acetate, PMA) for 12 h, and then the original medium was discarded, and 100 μL of new complete medium was added. Set the culture tone zero well without cells and the normal group without drugs; 1 μL of B4, A3, A3 derivative, dexamethasone (DEX), and celecoxib (CELE, 1 mM, 5 mM) were added to each well to a final drug concentration of 10 μM, 50 μM, and incubated in an incubator for a total of 24 h. After completion of incubation, 10 μL of CCK-8 was added to each well and incubated for 4 h in the dark. The absorbance of each well was measured with a microplate reader at a wavelength of 450 nm, and the cell viability rate of each well was calculated.

Calculation formula.

Cell viability (%)=[A(administered)−A(blank)]/[A(0 administered)−A(blank)]×100
A(administered): Absorbance of wells with cell, CCK-8, and drug solutions
A(blank): Absorbance of wells with medium and CCK-8 without cells
A(0 administered): Absorbance of wells with cells, CCK-8 without added drug

As shown in FIG. 8, the cytotoxicity of compound A3 and its derivatives was determined at concentrations of 10 and 50 μM, wherein the compounds of the present invention showed no significant cytotoxicity to THP-1 macrophages, and the abscissa in the figure represents the compound and the concentration, for example, A3-16-50 represents the derivative A3-16 at a concentration of 50 μM.

### Example 22

THP-1 cells were seeded in a 6-well plate at 2×10⁵ cells/well, and cultured conventionally. After the cells reached 80%, they were induced with 100 ng/mL PMA for 12 h and then the original medium was discarded, and new complete medium was added. The experimental group was pretreated with anemoside A3 and its derivatives 1 μM for 1 h, the control group was pretreated with the positive drugs dexamethasone 10 UM and celecoxib 10 μM for 1 h, and the control group was the blank group; After 1 h, the blank group was removed and the other groups were incubated with 1 μg/mL LPS for 2 h. Then the following procedure was performed on ice, the supernatant of the multi-well plate was removed, 4° C. precooled PBS was pipetted and gently added along the edges, and washed twice; After adding 1 mL of PBS, the cells were scraped with a cell scraper, placed in a 1.5 mL centrifuge tube, centrifuged at 2000 g at 4° C. for 3 min, the supernatant was discarded, and the cell pellet was collected. 100 μL of RIPA lysate (protease inhibitor and phosphatase inhibitor were added before use) was added to each tube, mixed well, and lysed on ice for 10 minutes. After the cells were disrupted again by the ultrasonic disruptor, centrifuged at 12000 g at 4° C. for 10 minutes, the cell supernatant was carefully collected in a new EP tube and stored on ice. With reference to the instructions, the BCA Protein Quantification Kit was used to measure and calculate the total protein content. After the protein sample was diluted with PBS, 5×SDS-PAGE loading buffer (50 μL β-mercaptocthanol has been added per mL) was added to achieve a final protein concentration of 2 μg/μL; It was boiled at 100° C. for 10 minutes to denature it to prevent protein degradation. SDS-PAGE gels with different concentrations were configured according to the required protein molecular weight, and the protein samples were loaded on the gels at a content of 20 mg/well for electrophoresis, and the separated protein samples were transferred to polyvinylidene fluoride (PVDF) membranes, and the PVDF membranes were washed 3 times with Tris-HCl buffered saline solution (TBST buffer) for 10 min each time. After it was blocked with the protein blocking solution for 1 hour at room temperature, and then was washed several times with TBST buffer until the clean blocking solution, and the PVDF membrane was incubated with the specific primary antibody overnight in a 4° C. refrigerator according to the instructions. On the next day, the PVDF membrane was thoroughly washed with TBST buffer, combined with the corresponding secondary antibody at room temperature for 1 hour, and thoroughly washed with TBST buffer again. Finally, the protein was exposed to color fraction with reference to the instructions of the special hypersensitive ECL chemiluminescence kit.

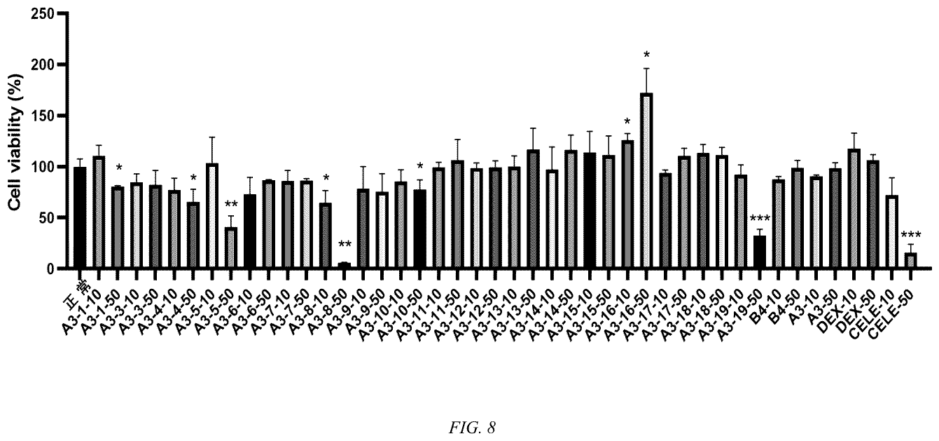

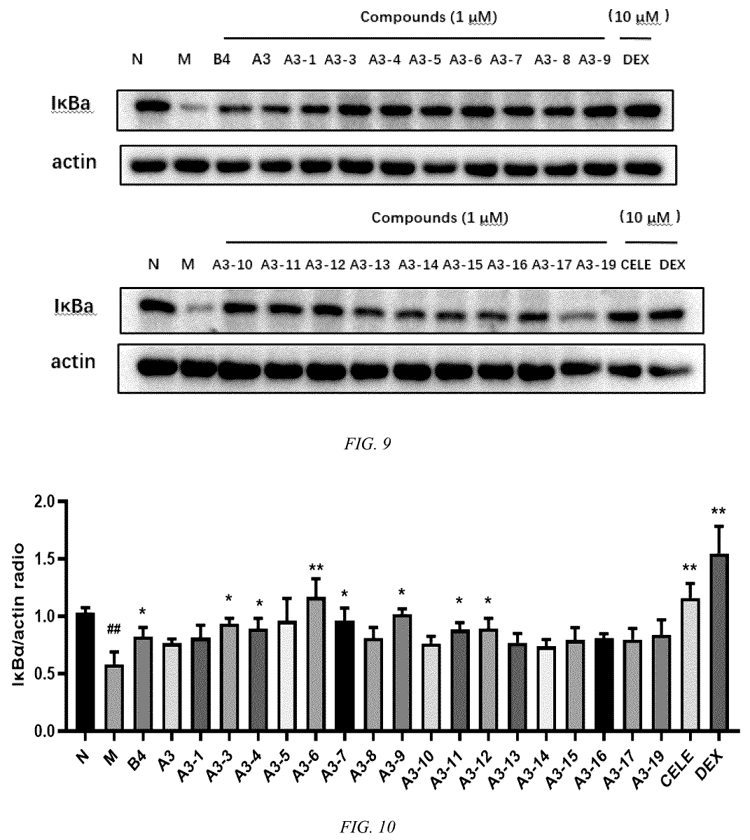

The prior art believes that anemoside B4 (B4) can inhibit the activation of key proteins in the NF-κB/MAPK signaling pathway. FIG. 9 showed a Western blotting strip, and FIG. 10 showed Western blotting to detect the expression of IκB protein. As shown in the figure, the level of IκB protein in THP-1 macrophages was displayed by Western blotting, and it was found that 2 h after THP-1 cells was stimulated with LPS, the level of IκB protein in the model group decreased significantly (P<0.01); Compared with the LPS model group, the compound of the present invention could increase the level of IκB protein, and the level of IκB protein of compound A3-6 was significantly increased compared with that of B4 (p<0.05). In all, these results suggest that A3-6 has better anti-inflammatory activity.

### Example 23

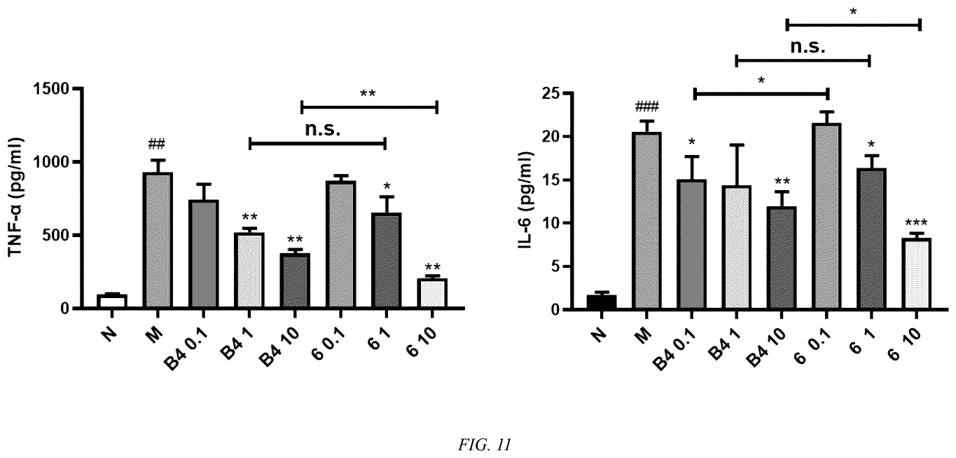

THP-1 cells were seeded in a 6-well plate at 2×10⁵ cells/well and cultured conventionally. After the cells reached 80%, they were induced with 100 ng/ml of PMA for 12 h, and then the original medium was discarded, and new complete medium was added. The control group was pretreated with B4 (0.1, 1, 10 μM) for 1 h, and the experimental group was pretreated with A3-6 (0.1, 1, 10 μM) for 1 h. After 1 h, the other groups were removed from the blank group and incubated with 1 μg/mL LPS for 24 h. The culture supernatant was collected, and the IL-6 and TNF-α contents were finally determined and calculated using ELISA kits step by step according to their instructions. The effect of A3-6 on the release of inflammatory factors was tested by ELISA kits. As shown in FIG. 11, after 24 h of LPS (1 μg/mL) stimulation, the release of IL-6 and TNF-α in the model group increased significantly (P<0.01), and the levels of the two inflammatory factors after administration of B4 and A3-6 (1, 10 μM) were significantly reduced compared with the model group (P<0.05), among which A3-6 (10 μM) was significantly different compared with B4 (10 μM) (P<0.05), proving that A3-6 had better anti-inflammatory effect compared with B4.

### Example 24: Therapeutic Effect of A3-6 on Colitis Mice

ICR mice were fed normally for 3 days, with free access to water and food during the feeding period. After weighing, the mice were randomly divided into groups, including normal group, model group (DSS group), A3-6 (15, 30, 60 mg/kg) group, A3-6 (60 mg/kg)+DADA (50 mg/kg) group, and positive drug B4 (100 mg/kg) group. A3-6 was administered by gavage with hydroxypropyl β-cyclodextrin (1:3), B4 was dissolved in water and administered by gavage, and DADA (50 mg/kg) was injected intraperitoneally, once a day. The day before modeling, the weight was weighed and A3-6, DADA and B4 were pre-administered according to body weight. After 24 hours of administration, DSS was dissolved in the drinking water for mice. The mice in the model group and the administration group were continuously given 5% DSS for 8 days and weighed every day. The fecal characteristics of mice were observed to check the occult blood/hematochezia of mice, and the DAI score was calculated. After the 9th day of administration, the mice were sacrificed, blood and colon were collected, and the length of the colon was measured.

A3-6 (15 mg/kg, 30 mg/kg and 60 mg/kg) or B4 (100 mg/kg) were administered by gavage once daily for 9 days. After 24 hours after administration, colitis was induced in mice with 5% DSS for eight consecutive days, and the mice were sacrificed on the ninth day after colitis induction. The DAI scores of each group of mice were calculated daily according to the scoring requirements.

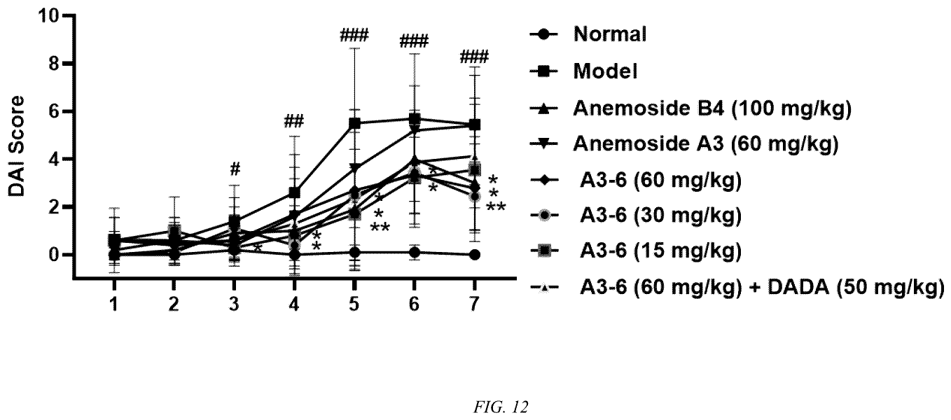

The mice colitis model was constructed using 5% DSS to evaluate the therapeutic effect of A3-6 on inflammatory bowel disease (IBD)-. The mice were continuously administered 5% DSS for 7 days. As a result, it was found that the mice were depressed, the feces were soft and shapeless, and occult blood or hematochezia were present on the 4th day of DSS modeling. On the 6th day of modeling, the mice developed watery feces and severe hematochezia. The DAI score of the mice was comprehensively evaluated. As shown in FIG. 12, from the fourth day of modeling, the colitis condition of the mice in the DSS model group began to worsen, and the DAI score increased, and reached the peak on the sixth day. The DAI score of mice decreased significantly on the 4th day after administration of A3-6 15 mg/kg, which was significantly different from that of mice in the model group (P<0.05). The DAI score of mice in the A3-6 (30 mg/kg) group decreased significantly from the 5th day, which was significantly different from that in the model group (P<0.05). The DAI score of mice in the A3-6 (60 mg/kg) group decreased significantly from the 7th day, which was significantly different from that in the model group (P<0.05). However, there was no significant difference between the A3-6 (60 mg/kg)+DADA (50 mg/kg) group and the model group, indicating that DADA (50 mg/kg) had a reversal effect on the DAI score of colitis mice in the A3-6 treatment group.

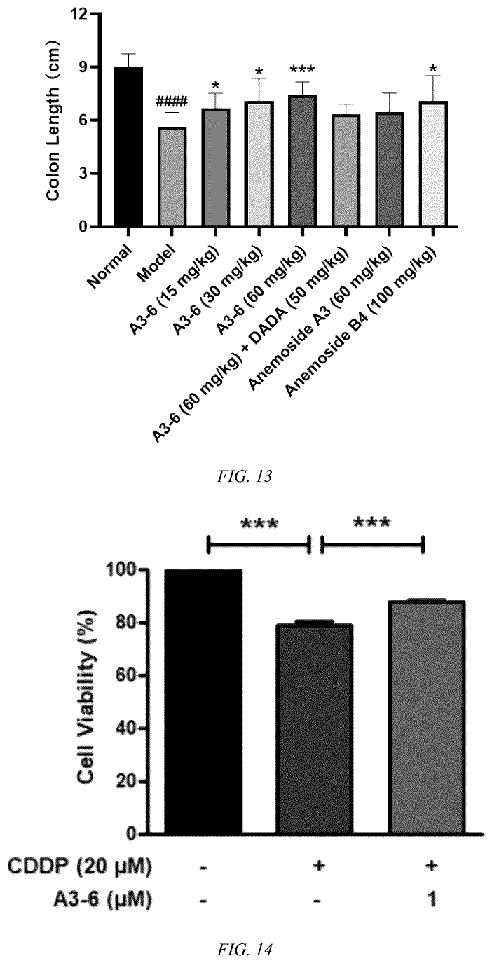

As shown in FIG. 13, the colon length of the DSS model mice was significantly shorter than that of the normal control group (P<0.0001). A3-6 15 mg/kg, 30 mg/kg, 60 mg/kg groups and B4 group could restore the colon length of mice. The dose groups of A3-6 15 mg/kg, 30 mg/kg, 60 mg/kg groups and B4 group could restore the colon length of mice. The above experimental results showed that the dose groups of A3-6 15 mg/kg, 30 mg/kg, and 60 mg/kg could alleviate the symptoms of colitis in mice.

### Example 25

HEK-293T cells were seeded in a 6-well plate at 2×10⁵ cells/well, cultured conventionally, pretreated with A3-6 (1 μM) for 1 h, stimulated with 20 μM cisplatin for 24 h, the original cell culture medium was discarded, and the cells were washed with serum-free DMEM medium. The peroxide-sensitive fluorescent probe 2,7-dichlorofluorescein diacetate (DCFH-DA) was diluted with serum-free DMEM medium according to 1:1000 to make the final concentration 10 μM, and 1 mL of serum-free medium containing DCFH-DA probe was added to cach well, away from light, and incubated in a 5% CO₂, 37° C. incubator for 30 minutes, and gently shaken several times every 10 minutes to fully contact the fluorescent probe and cells. Discard the original culture medium, digest with 0.25% trypsin digestion solution (without EDTA), terminate the digestion with DMEM medium containing 10% FBS immediately after the cells fall off, transfer the cell suspension to a 10 mL centrifuge tube, centrifuge at 1000 rpm/min for 8 minutes, obtain cell pellets, and then wash the cells with DMEM medium without FBS to achieve the purpose of removing the fluorescent probe DCFH-DA that has not entered the cells. Finally, protect it from light and test it by flow cytometry.

HEK-293T cells in the logarithmic growth phase, with good cell condition and appropriate density were taken to obtain a cell suspension, and the cell density was adjusted to 5×10⁴ cells/mL with complete medium, the cells were mixed uniformly and seeded in a 96-well plate at 100 μL/well, and a row of wells was reserved without cells and only the complete medium was added as a blank control, and cultured in an incubator at 37° C.; When the confluence reached 70%-80%, A3-6 (final concentration of 1 μM) was administered to the treatment group and the same volume of media was administered to the normal group. Cells in the model and treatment groups were incubated with cisplatin at a final concentration of 20 μM 1 h after dosing. After 24 h, CCK-8 kit was used to test the cell viability rate.

The main feature of cisplatin nephrotoxicity is that it causes kidney cell damage and apoptosis, so the protective effect of A3-6 on human embryonic kidney (HEK 293T) cells was tested using CCK-8 kit. As shown in FIG. 14, the results showed that the survival rate of cells increased from 79% to 87% when the concentration of A3-6 was 1 μM (P<0.001); The above experimental results show that A3-6 antagonizes the cytotoxicity of cisplatin.

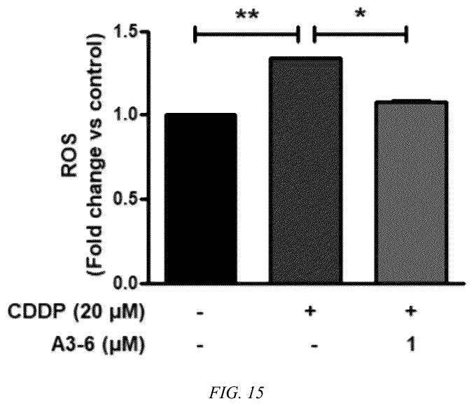

Meanwhile, cisplatin-induced diseases are closely associated with elevated levels of reactive oxygen species (ROS). As shown in FIG. 15, the ROS level in HEK-293T cells labeled with DCFH-DA probe was tested and analyzed by flow cytometry, and it was found that after cisplatin stimulated kidney cells for 24 h, the ROS level increased to 1.5 times that of the normal control group (P<0.01); Compared with the cisplatin model group, the ROS level was significantly reduced after 1 μM A3-6 was treated with cells 1 h in advance, and there was a significant difference (P<0.05). The above results indicate that A3-6 can significantly inhibit the release of reactive oxygen species (ROS) in renal cells induced by cisplatin.

1. A derivative of pentacyclic triterpenoidal saponin having the following chemical structure formula: 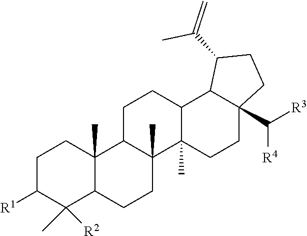
R¹ is hydrogen, hydroxy, halogen, C₁₋₈ alkoxy or —O-T; wherein T is a monosaccharide or a disaccharide; R² is alkyl, haloalkyl, or oxygen-substituted alkyl; R³ is oxygen, hydroxy or hydrogen; R⁴ is —NHR^{a}, —N(R^{a})₂, or —OR^{b}, wherein R^{a} is hydrogen, alkyl, aryl-C₁₋₆ alkyl, hydroxy-C₁₋₆ alkyl or amino-C₁₋₆ alkyl, aromatic heterocycle, amino acid or amino acid ester; R^{b} is alkyl, aryl-C₁₋₆ alkyl, hydroxy-C₁₋₆ alkyl, amino-C₁₋₆ alkyl, cycloalkyl, aromatic heterocycle, Alkyl ester, amino acid or amino acid ester.

2. The derivative of pentacyclic triterpenoidal saponin according to claim 1, wherein the monosaccharide is a C₄₋₇ monosaccharide.

3. The derivative of pentacyclic triterpenoidal saponin according to claim 1, wherein the monosaccharide includes glucose, arabinose, rhamnose, galactose or xylose or acetylated forms; disaccharide includes α-L-rhamnopyranosyl-(1→2)-α-L-arabinopyranosyl or acetylated forms.

4. The derivative of pentacyclic triterpenoidal saponin according to claim 1, wherein the amino acid is glycine, aminobutyric acid, aminocaproic acid, phenylalanine, alanine, cysteine, leucine or serine; the amino acid ester is ethyl glycine, ethyl aminobutyrate, methyl aminocaproate, ethyl phenylalanine, ethyl alanine, ethyl cysteine, ethyl leucine, or ethyl serine.

5. The derivative of pentacyclic triterpenoidal saponin according to claim 1, wherein the derivative of pentacyclic triterpenoidal saponin is prepared by esterification or amidation reaction using compound A3 as a raw material.

6. A pharmaceutical composition comprising the derivative of pentacyclic triterpenoidal saponin according to claim 1 as an active ingredient.

7. An application of the derivative of pentacyclic triterpenoidal saponin according to claim 1 in the preparation of anti-inflammatory drugs, antioxidant drugs and anti-apoptotic drugs.

8. An application of the derivative of pentacyclic triterpenoidal saponin according to claim 1 in the preparation of a medicament for the treatment of inflammatory bowel disease.

9. An application of the derivative of pentacyclic triterpenoidal saponin according to claim 1 in the preparation of a medicament for alleviating nephrotoxicity of cisplatin.

10. The application according to claim 7, wherein the medicament is a topical, oral, rectal or parenteral medicament.
